(19) United States
(12) Patent Application Publication
(10) Pub. No.: US 2024/0197867 A1
(43) Pub. Date: Jun. 20, 2024

(54) NEURAL CONTROL OF ADAPTIVE IMMUNITY

(71) Applicant: The Feinstein Institutes for Medical Research, Manhasset, NY (US)

(72) Inventors: Sangeeta S. Chavan, Syosset, NY (US); Kevin J. Tracey, Old Greenwich, CT (US)

(21) Appl. No.: 18/278,688

(22) PCT Filed: Feb. 25, 2022
(86) PCT No.: PCT/US2022/017819

§ 371 Date: Aug. 24, 2023

Related U.S. Application Data
(60) Provisional application No. 63/154,338, filed on Feb. 26, 2021.

(51) Int. Cl. A61K 39/39; A61K 39/00

(52) U.S. Cl. CPC A61K 39/39; A61K 2039/55511

(57) ABSTRACT

The present invention provides methods for modulating antigen-specific antibody responses by administering to a mammal a compound that selectively either activates or inhibits a specific subset of sensory neurons that express TRPV1.

### CROSS-REFERENCE TO RELATED APPLICATION

This application is a National Phase of International Application No. PCT/US2022/017819, filed Feb. 25, 2022, which claims benefit and priority to U.S. Provisional Application No. 63/154,338, filed Feb. 26, 2021, which are incorporated herein by reference in their entirety.

### FIELD OF THE INVENTION

The present invention provides a method for activation or inhibition of sensory neurons using pharmacological agents or neuromodulation techniques that can be used for the modulation of antigen-specific antibody responses. Hence, the present invention can be used in the context of vaccination and/or in treating allergic or autoimmune disorders.

### BACKGROUND OF THE INVENTION

The nervous system perceives the environment by receiving input from a variety of types of sensory neurons. Sensory neurons of one such class are the pain-sensing neurons, termed “nociceptors”. These specialized neurons densely innervate most peripheral tissues, and detect various noxious stimuli such as chemicals, temperature, and pathogens. In the densely innervated lungs, sensory neurons detect novel antigens and respond to these stimuli. Using neuromodulation technologies that act directly upon nerves and are restricted to specific neural populations, it is possible to regulate and to shape effective antigen-specific immune responses against novel antigens within the lung. Adaptive immune responses are the basis of vaccine efficacy. Hence, therapeutic manipulation of specific neural fibers in humans are a tractable target for use in the clinical settings of vaccination and/or in treating allergies or autoimmune disorders.

The lung is densely innervated by sensory neurons expressing nociceptors including the transient receptor potential vanilloid channel 1 (hereafter “TRPV1”). Nociceptors detect and respond to pathogen-associated molecular pattern and other inflammatory mediators. However, the function of nociceptors in regulating lung antigen-specific responses is unknown.

### SUMMARY OF THE INVENTION

The present invention provides methods for regulating antigen-specific antibody responses by administering to a mammal a compound that selectively either activates or inhibits a specific subset of sensory neurons that express TRPV1.

The methodology described herein can be used for improving vaccination or inhibiting antibody responses to specific antigens. According to the present invention, this activation or inhibition may be achieved by any compound or any neurostimulation technique that either activates or suppresses TRPV1-expressing neurons.

### BRIEF DESCRIPTION OF THE DRAWINGS

FIG. 1. Silencing of sensory nerves at the time of primary immunization impacts antigen specific antibody responses. TRPV1^{Cre}/Gi-DREADD mice were administered with vehicle (DMSO) or CNO 30 min prior to immunization. Titer of NP-specific antibodies were monitored using ELISA.

FIG. 2. Selective optogenetic activation of TRPV1-expressing neurons enhances antigen-specific antibody response following immunization. TRPV1-ChR2 mice or wild-type mice were subjected to optogenetic stimulation using blue light on the dorsum of the hind paw immediately prior to immunization in the same paw with KLH. Serum was collected every 7 days and assayed for anti-KLH IgG antibodies by ELISA.

### DETAILED DESCRIPTION OF THE INVENTION

This invention provides for a method of providing a vaccine adjuvant in a subject who is being administered an immunogen-specific vaccine, which comprises administering to said subject an immunogen-specific antibody production enhancing amount of a TRPV1 nociceptor agonist (alternatively termed herein a TRPV1 agonist or a TRPV1 receptor agonist), thereby providing a vaccine adjuvant in the subject. This invention also provides for a method of increasing antibody production in a subject to at least one antigen contained in a vaccine that is being administered to said subject, which comprises administering to said subject an antibody-production increasing amount of a TRPV1 nociceptor agonist.

Another embodiment of the present invention is the method identified in the embodiments above wherein the TRPV1 receptor agonist is administered concurrently with the vaccine. In an alternative embodiment, the TRPV1 receptor agonist is administered after administration of the vaccine.

In an embodiment, the TRPV1 receptor agonist can be administered using a variety of methods known in the art. More particularly, the TRPV1 receptor agonist can be administered by any suitable route of administration, including administration methods such as topical, parenteral, intramuscular, nasal, oral, transdermal, mucosal, and subcutaneous. or other modes of delivery known in the art.

In an embodiment of the present invention the TRPV1 receptor agonist is administered topically in the same general body area of the subject as the vaccine. Alternatively, the TRPV1 receptor agonist is administered systemically.

Provided methods are useful for the treatment and/or prevention of microbial infections, such as infections caused by bacteria, viruses, fungi and parasites. In an embodiment of the present invention the immunogenic-specific vaccine immunizes against, for example, disease states such as, but not limited to, measles, hepatitis B, meningitis, mumps, pertussis, poliomyelitis, rubella, tetanus, tuberculosis, yellow fever, influenza, or COVID-19.

As used herein, the terms “treat”, “treating”, “treatment” and the like, are meant to decrease, suppress, attenuate, diminish, arrest, the development or progression of the microbial infection and/or symptoms associated therewith. The terms “treat”, “treating”, “treatment” and the like, as used herein can refer to curative therapy, prophylactic therapy, and preventative therapy. Accordingly, as used herein, “treating” means either slowing, stopping or reversing the progression of the microbial infection, including reversing the progression to the point of eliminating the symptoms of the microbial infection.

As used herein, the terms “prevent”, “preventing”, “prevention”, “prophylactic treatment” and the like refer to reducing or inhibiting the probability of developing symptoms of the microbial infection in a subject. Thus, in some embodiments, a TRPV1 receptor agonist can be administered prophylactically to prevent the onset of microbial infections or to prevent the recurrence of microbial infections in a subject.

The subject of the inventive methods may be a human or a non-human animal. Non-limiting examples of non-human animals that may be used in the methods according to the invention include companion animals, such as dogs or cats, and large animals such as those used in animal husbandry (for example, sheep, cattle, pigs, llamas, buffalo etc.) or wild animals such as tigers, lions, elephants, etc.

In an embodiment, adjuvant compositions are provided comprising a TRPV1 receptor agonist that enhances the immune response to the one or more antigens/immunogens. “Adjuvant” as used herein refers to an agent that, when present in an effective amount, increases the antigenic response; a substance enhancing the immune response to an antigen; or an agent that stimulates antibody production to an antigen. The administration of such adjuvant compositions is intended to induce the activity of the TRPV1 nociceptor in the subject to be treated.

In another embodiment, an immunogenic composition comprising a TRPV1 receptor agonist and an antigen are provided. Said immunogenic compositions may be used as a vaccine. The antigen, for purposes of the immunogenic composition, can be any antigen or combination of antigens suitable to induce an immunogenic response in a subject. In some preferred forms, the antigen can be either a modified-live or killed microorganism, or a natural product purified from a microorganism or other cell including, but not limited to, a tumor cell, a synthetic product, a genetically engineered protein, peptide, polysaccharide or similar product, or an allergen. The antigen can also be a subunit of a protein, peptide, polysaccharide or similar product. The antigen may also be the genetic antigens, i.e., the DNA or RNA that engenders an immune response. Representative of the antigens that can be used according to the present invention include, but are not limited to, natural, recombinant or synthetic products derived from viruses, bacteria, fungi, parasites and other infectious agents in addition to autoimmune diseases, hormones, or tumor antigens which might be used in prophylactic or therapeutic vaccines and allergens. Such compositions find use in, among other things, clinical (e.g., therapeutic and preventative medicine (e.g., vaccination)) and research applications.

Adjuvant compositions and immunogenic compositions may be formulated with pharmaceutically acceptable carriers. As used herein, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintigrants (e.g., potato starch or sodium starch glycolate), polyethylethe glycol, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

Non-limiting examples of the TRPV1 receptor agonists that can be used in the present invention include capsaicin, resiniferatoxin (RXT), eugenol, camphor, clotrimazole, arvanil (N-arachidonoylvanillamine), anandamide, phorbol 12-phenylacetate 13-acetate 20 homovanillate (PPAHV), olvanil (NE 19550), N-oleoyldopamine (OLDA), 6′-iodoresiniferatoxin (6′IRTX), C18 N-acylethanolamines (NAE's), lipoxygenase derivatives, 12-hydroperoxyeicosatetraenoix acid (HETE), inhibitor cysteine knot (ICK) peptides (vanillotoxins), piperine, (N-[2-(3,4-dimethylbenzyl)-3-(pivaloyloxy)propyl)-2-[4-(2-amioethoxy)-3-methoxyphenyl]acetamide) (MSK195), (N-[2-(3,4-dimethylbenzyl)-3-pivaloyloxy)propyl]-N′-(4-hydroxy-3-methoxybenzyl)thiourea (JYL79), hydroxy-alpha-sanshool, 2-aminoethoxydiphenyl borate, 10-shogaol, oleylgingerol, oleylshogaol, (N-(4-tert-butylbenzyl)-N′-(4-hydroxy-3-methoxybenzyl)thiourea (SU200), amylocaine, articaine, benzocaine, bupivacaine, carbocaine, caricaine, chloroprocain, cyclomethycaine, dibucaine (chinchocaine), dimethocaine (larocaine), ethidocaine, hexylcaine, levobupivacaine, lidocaine, mepivacaine, meprylcaine (orocaine), metautoxycaine, piperocaine, pricocaine, procaine (novocaine), proparacaine, propoxycaine, risocaine, popicovaine, tetracaine (amethocatine), trimencaine, fatty acid amides, 12-lipoxygenase-derivated eicosanoids, black pepper compounds, other pepper components, vannilloids (n-vallinyl-alkanedienamides, N-vanillyl-alkanedienyls, N-vanillyl-cis-monosaturated alkenamides, capsiate, dihydrocapsiate, nordihydrocapsisates, capsinoids, capsiconiate, dihydrocapsiconiates, coniferyl esters, a capsiconinoid, a ricinolenic acid derivative, tinyatoxin, civamide, N-[4-(2-aminoethoxy)-3-methoxyphenyl)-methyl]-9Z-octa-decanamide, triprenyl phenols (e.g., scutigeral), piperines, zingerone, NE-21610, and NE-28345.

Capsiconinoids include compounds such as:

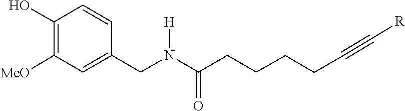

wherein R is a substituent selected from the group consisting of alkyl, alkenyl, alkynyl, allyl, aryl, aalkoxy, aryloxy, alkanoyl, aroyl, amino, alkylthio, arylthiol, cyano, cycloalkyl, cycloalkenyl, halo, hydoxy, oxo, nitro, and trifluoromethyl;
when said substituent R contains a carbon chain, it may be straight-chained or branched and possibly further substituted with alkyl, alkenyl, alkynyl, allyl, aryl, alkoxy, aryloxy, alkanoyl, aroyl, amino, alkylthio, arylthio, cyano, cycloalkyl, cycloalkenyl, halo, hydroxy, oxo, nitro, or trifluoromethyl,
   which are provided for in U.S. Pat. No. 7,446,226, herein incorporated by reference.

Ricinoleic acid derivatives include compounds such as:

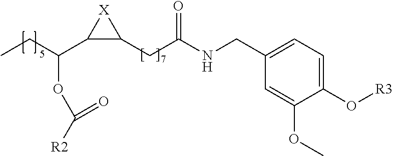

in which
X represents two hydrogen atoms, a x-bond, oxygen, or methylene;
R2 is a C₆-C₁₂ aryl or arylalkyl residue; and
R3 is hydrogen, 2-hydroxyethyl, or 2-aminoethyl,
   which are provided for in U.S. Pat. No. 7,429,673, herein incorporated by reference.

This invention further provides for a method of treating allergic or autoimmune disorders in a subject, comprising the step of administering to said subject a TRPV1 receptor antagonist. In an embodiment, a method of treating, preventing the progression of, or delaying the onset of allergic or autoimmune disorders in a subject is provided, the method comprising administering to the subject a therapeutically effective amount of at least one TRPV1 receptor antagonist. In one embodiment, such treatments are designed to reduce the activity of TRPV1 in the subject to be treated.

Such “allergic” disorders include, but are not limited to, asthma, respiratory allergy (e.g., pollen allergy), stinging insect allergy, allergic rhinitis (hay fever), food allergy, drug allergy, latex allergy, atopic dermatitis (eczema), or other allergic disease known in the art.

Such “autoimmune disorders” include, but are not limit to, Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia, sympathetic ophthalmia, myasthenia gravis, Graves' disease, primary biliary cirrhosis, chronic aggressive hepatitis, membranous glomerulopathy, reactive arthritis (including Reiter's syndrome), polymyositis-dermatomyositis, systemic sclerosis, polyarteritis nodosa, multiple sclerosis, bullous pemphigoid, Cogan's syndrome, ankylosing spondylitis, Wegener's granulomatosis, autoimmune alopecia, and inflammatory bowel diseases, such as Crohn's disease, collagenous colitis, granulomatous ileocolitis, idiopathic inflammatory bowel disease, ileitis, irritable bowel syndrome, lymphocytic colitis, regional enteritis, spastic colon, and ulcerative colitis. In particular embodiments, the methods may be used to treat or prevent inflammatory bowel diseases, such as Crohn's disease, collagenous colitis, granulomatous ileocolitis, idiopathic inflammatory bowel disease, ileitis, irritable bowel syndrome, lymphocytic colitis, regional enteritis, spastic colon, and ulcerative colitis. In some examples, the inflammatory bowel disease is ulcerative colitis, Crohn's disease, lymphocytic colitis, or collagenous colitis.

As used herein, the terms “treat”, “treating”, “treatment” and the like, are meant to decrease, suppress, attenuate, diminish, arrest, the underlying cause of the allergic or autoimmune disorder or to stabilize the development or progression of the allergic or autoimmune disorder and/or symptoms associated therewith. The terms “treat”, “treating”, “treatment” and the like, as used herein can refer to curative therapy, prophylactic therapy, and preventative therapy. Accordingly, as used herein, “treating” means either slowing, stopping or reversing the progression of the allergic or autoimmune disorder, including reversing the progression to the point of eliminating the symptoms of the allergic or autoimmune disorder.

As used herein, the terms “prevent”, “preventing”, “prevention”, “prophylactic treatment” and the like refer to reducing or inhibiting the probability of developing symptoms of an allergic or autoimmune disorder in a subject, who does not have, but is at risk of or susceptible to developing an allergic or autoimmune disorder. Thus, in some embodiments, a TRPV1 receptor antagonist can be administered prophylactically to prevent the onset of an allergic or autoimmune disorder or to prevent the recurrence of an allergic or autoimmune disorder in a subject.

In an embodiment, pharmaceutical compositions are provided comprising a TRPV1 receptor antagonist (alternatively termed herein a TRPV1 antagonist or a TRPV1 nociceptor antagonist) that acts as an agent for treatment of an allergic or autoimmune disorder. Said pharmaceutical compositions comprise one or more TRPV1 receptor antagonists and a pharmaceutically acceptable carrier. As stated above, the term “pharmaceutically acceptable carrier” refers to any of the standard pharmaceutical carriers including, but not limited to, phosphate buffered saline solution, water, and various types of wetting agents (e.g., sodium lauryl sulfate), any and all solvents, dispersion media, coatings, sodium lauryl sulfate, isotonic and absorption delaying agents, disintigrants (e.g., potato starch or sodium starch glycolate), polyethylethe glycol, and the like. The compositions also can include stabilizers and preservatives. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference). Non-limiting examples of the TRPV1 antagonists that can be used in the present invention include capsazepine (N-[2-(4-chlorophenyl)ethyl]-7,8-dihydroxy-1,3,4,5-tetrahydro-2H-benzazepine-2-carbothioamide), azo-capsazepines (AC4), thio-BCTC, BCTC, and ruthenium red.

In an embodiment of the present invention, the TRPV1 receptor antagonist can be administered using a variety of methods known in the art. More particularly, as described herein, the TRPV1 nociceptor antagonist (or any of the forementioned TRPV1 agonists) can be administered by any suitable route of administration, including orally, nasally, transmucosally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments, including buccally and sublingually, transdermally, through an inhalation spray, or other modes of delivery known in the art.

In yet another embodiment, kits comprising a TRPV1 receptor agonists for stimulating an antibody-specific antibody response are provided. Such kits contain materials useful for administering to a subject an immunogen-specific antibody production enhancing amount of a TRPV1 nociceptor agonist, thereby providing a vaccine adjuvant in the subject. as described herein. In another embodiment the kit may further comprise specific antigens for which immunization is desired. The kits may comprise one or more of the following components: a container and a label or package insert on or associated with the container. Suitable containers include, for example, bottles, vials, syringes, IV solution bags, etc. Instructions for use of the kit may also be included.

In yet another embodiment, kits comprising a TRPV1 receptor antagonists for treatment of an allergic or autoimmune disorder are provide. Such kits contain materials useful for the treatment of an allergic or autoimmune disorder as described herein. The kits may comprise one or more of the following components: a container and a label or package insert on or associated with the container. Suitable containers include, for example, bottles, vials, syringes, IV solution bags, etc.

Although the present invention and its advantages have been described in detail, it should be understood that various changes, substitutions, and alterations can be made herein without departing from the spirit and scope of the invention as defined in the claims below.

The present invention will be further illustrated in the following Examples, which are given for illustration purposes only and are not intended to limit the invention in any way

### EXAMPLES

### Methods

Using an intra-tracheal instillation technique, specific antigens were delivered in the lung. Tracheal instillations are preferred methodology, over inhalation protocols that may primarily target the upper respiratory tract. This allows for making subsequent physiological measurements on test animals, or reinstallation using the same animal. Also, the dose of the inoculum introduced into the lungs can be carefully determined and more accurately delivered to ensure animal recovery. Next, using chemogenetic technology, which enables temporal control of neural activity using Designer Receptors Exclusively Activated by Designer Drugs (DREADD), it was demonstrated that TRPV1-expressing neurons are required for mounting an efficient antigen-specific antibody response. TRPV1+ neurons were selectively silenced at the time of immunization using a chemogenetic mouse model. The Gi-DREADD, also known as human muscarinic receptor 4 (hM4Di), is a mutated muscarinic acetylcholine receptor that, when activated by its ligand, CNO, initiates the inhibitory G-protein signaling cascade that inhibits neuronal signaling and/or excitability. TRPV1Cre/Gi-DREADD mice that express Gi-DREADD on TRPV1+ neurons were generated by crossing TRPV1Cre mice with R26-LSLGi-DREADD mice controls. TRPV1Cre/Gi-DREADD mice were administered with a small-molecule chemical (e.g., clozapine N-oxide (“CNO”)) intraperitoneally to antagonize modified muscarinic acetylcholine receptors expressed in relevant neuronal targets in vivo prior to or after immunization as described above. Antibody levels were assessed over time. The results demonstrated that specific inhibition of TRPV1-expressing neurons abrogates antigen-specific antibody levels.

An optogenetic approach to selectively activate TRPV1-expressing neurons during immunization in order to permit one to assess the potential TRPV1-expressing nociceptor to immune cell signaling axis without a priori knowledge of the corresponding molecular or signaling mechanisms. Optical control of a neuron was achieved by targeted expression of a light-gated cation channel (e.g., channelrhodopsin-2 (“ChR2”)), in a selective population of neurons, thereby allowing temporally precise modulation of targeted neuronal activity. To specifically activate TRPV1-expressing neurons during immunization, TRPV1-Cre/ChR2 mice were used in which TRPV1 lineage neurons expressed ChR2, generated by crossing TRPV1-Cre mice with Rosa26ChR2-eYFP/+ (ChR2) mice. To directly test changes in antigen-specific antibody responses induced by ChR2-mediated TRPV1 excitation, dorsal paw innervating TRPV1-expressing sensory neurons in TRPV1-Cre/ChR2 mice was optically stimulated with blue light (450-490 nm) directed through a LED source during antigenic challenge. C57BL/6 mice devoid of ChR2 were exposed to identical stimulation parameters to control for the light exposure procedure. Following light stimulation and immunization, serum anti-KLH antibody levels were monitored in mice at day 14 and 28 post-immunization. Optogenetic stimulation of TRPV1+ neurons significantly increased antigen-specific antibody responses in TRPV1-Cre/ChR2 mice as compared to wild-type mice.

### Example 1

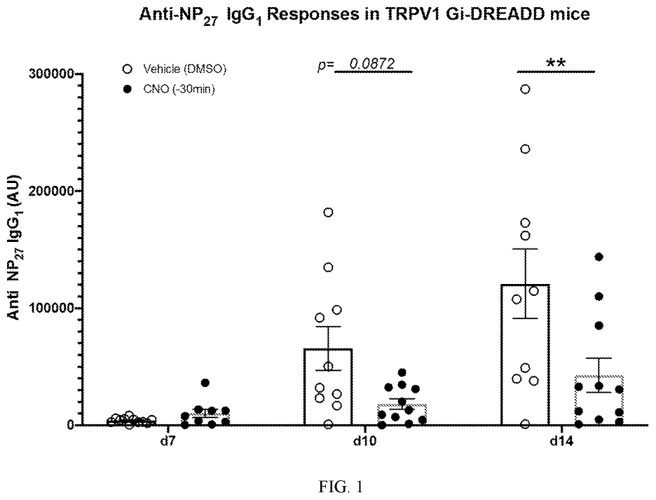

Using chemogenetic mice it was demonstrated that sensory nociceptor fibers expressing TRPV1 are required for efficient antigen-specific immune responses. Chemogenetics enable temporal control of neuron activity using Designer Receptors Exclusively Activated by Designer Drugs (DREADD). Inhibitory G protein (Gi-DREADD) is a modified muscarinic acetylcholine receptor that binds the ligand, clozapine-N-oxide (CNO), which triggers inhibitory G protein signaling to inhibit neuronal excitability. To selectively silence TRPV1⁺ nociceptors, we generated TRPV1-Gi-DREADD mice to transiently inhibit neuronal activity specifically in TRPV1⁺ neurons (See FIG. 1). CNO or Vehicle was administered intraperitoneally to TRPV1-Gi-DREADD mice 30 minutes prior to intratracheal immunization with 4-Hydroxy-3-nitrophenylacetyl hapten coupled to ovalbumin (NP₂₇-OVA). Chemogenetic inhibition of TRPV1⁺ neurons, before primary immunization, significantly diminishes NP-specific antibody responses. A significant reduction in NP-specific IgG in mice receiving CNO as compared to vehicle controls at 14 days post-immunization (Anti-NP IgG₁ U/mL, Mean±SEM, D14: vehicle, 120,877+29,519, n=10 versus CNO, 42,525+14,641, n=11, ** p=0.0017).

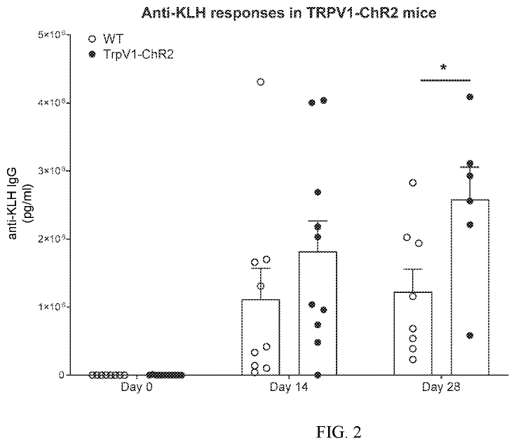

FIG. 2 illustrates that selective optogenetic activation of TRPV1-expressing neurons enhanced the antigen-specific antibody response following immunization. TRPV1-ChR2 mice or wild-type mice were subjected to optogenetic stimulation using blue light on the dorsum of the hind paw immediately prior to immunization in the same paw with KLH. Serum was collected every 7 days and assayed for anti-KLH IgG antibodies by ELISA.

These results indicate TRPV1 sensory neurons are required to initiate primary antibody responses to novel antigens. These findings offer significant new insights into the biological mechanisms of vaccination.

Having thus described in detail preferred embodiments of the present invention, it is to be understood that the invention defined above is not limited to particular details set forth in the above description as many apparent variations thereof are possible without departing from the spirit or scope of the present invention.

What is claimed is:

1. A method of providing a vaccine adjuvant in a subject who is being administered an immunogen-specific vaccine, which comprises administering to said subject an immunogen-specific antibody production enhancing amount of a TRPV1 receptor agonist, thereby providing a vaccine adjuvant in the subject.

2. The method according to claim 1, wherein the TRPV1 receptor agonist is selected from the group consisting of capsaicin, resiniferatoxin (RXT), eugenol, camphor, clotrimazole, arvanil (N-arachidonoylvanillamine), anandamide, phorbol 12-phenylacetate 13-acetate 20 homovanillate (PPAHV), olvanil (NE 19550), N-oleoyldopamine (OLDA), 6′-iodoresiniferatoxin (6′IRTX), C18 N-acylethanolamines (NAE's), lipoxygenase derivatives, 12-hydroperoxyeicosatetraenoix acid (HETE), inhibitor cysteine knot (ICK) peptides (vanillotoxins), piperine, (N-[2-(3,4-dimethylbenzyl)-3-(pivaloyloxy)propyl)-2-[4-(2-amioethoxy)-3-methoxyphenyl]acetamide) (MSK195), (N-[2-(3,4-dimethylbenzyl)-3-pivaloyloxy)propyl]-N′-(4-hydroxy-3-methoxybenzyl)thiourea (JYL79), hydroxy-alpha-sanshool, 2-aminoethoxydiphenyl borate, 10-shogaol, oleylgingerol, oleylshogaol, (N-(4-tert-butylbenzyl)-N′-(4-hydroxy-3-methoxybenzyl)thiourea (SU200), amylocaine, articaine, benzocaine, bupivacaine, carbocaine, caricaine, chloroprocain, cyclomethycaine, dibucaine (chinchocaine), dimethocaine (larocaine), ethidocaine, hexylcaine, levobupivacaine, lidocaine, mepivacaine, meprylcaine (orocaine), metautoxycaine, piperocaine, pricocaine, procaine (novocaine), proparacaine, propoxycaine, risocaine, popicovaine, tetracaine (amethocatine), trimencaine, fatty acid amides, 12-lipoxygenase-derivated eicosanoids, black pepper compounds, other pepper components, vannilloids (n-vallinyl-alkanedienamides, N-vanillyl-alkanedienyls, N-vanillyl-cis-monosaturated alkenamides, capsiate, dihydrocapsiate, nordihydrocapsisates, capsinoids, capsiconiate, dihydrocapsiconiates, coniferyl esters, a capsiconinoid, a cicinoleic acid derivative, tinyatoxin, civamide, N-[4-(2-aminoethoxy)-3-methoxyphenyl)-methyl]-9Z-octa-decanamide, triprenyl phenols, piperines, zingerone, NE-21610, and NE-28345.

3. The method according to claim 1, wherein the TRPV1 receptor agonist is administered concurrently with the vaccine.

4. The method according to claim 1, wherein the TRPV1 receptor agonist is administered topically in the same general body area of the subject as the vaccine.

5. The method according to claim 1, wherein the TRPV1 receptor agonist is administered systemically.

6. The method according to claim 1, wherein the subject is a human.

7. The method according to claims 1, wherein the subject is a non-human animal.

8. The method according to claim 1, wherein the immunogenic-specific vaccine immunizes against measles, hepatitis B, meningitis, mumps, pertussis, poliomyelitis, rubella, tetanus, tuberculosis, yellow fever, influenza, or COVID-19.

9. A method of increasing antibody production in a subject to at least one antigen contained in a vaccine that is being administered to said subject, which comprises administering to said subject an antibody-production increasing amount of a TRPV1 receptor agonist.

10. The method according to claim 9, wherein the TRPV1 receptor agonist is selected from the group consisting of capsaicin, resiniferatoxin (RXT), eugenol, camphor, clotrimazole, arvanil (N-arachidonoylvanillamine), anandamide, phorbol 12-phenylacetate 13-acetate 20 homovanillate (PPAHV), olvanil (NE 19550), N-oleoyldopamine (OLDA), 6′-iodoresiniferatoxin (6′IRTX), C18 N-acylethanolamines (NAE's), lipoxygenase derivatives, 12-hydroperoxyeicosatetraenoix acid (HETE), inhibitor cysteine knot (ICK) peptides (vanillotoxins), piperine, (N-[2-(3,4-dimethylbenzyl)-3-(pivaloyloxy)propyl)-2-[4-(2-amioethoxy)-3-methoxyphenyl]acetamide) (MSK195), (N-[2-(3,4-dimethylbenzyl)-3-pivaloyloxy)propyl]-N′-(4-hydroxy-3-methoxybenzyl)thiourea (JYL79), hydroxy-alpha-sanshool, 2-aminoethoxydiphenyl borate, 10-shogaol, oleylgingerol, oleylshogaol, (N-(4-tert-butylbenzyl)-N′-(4-hydroxy-3-methoxybenzyl)thiourea (SU200), amylocaine, articaine, benzocaine, bupivacaine, carbocaine, caricaine, chloroprocain, cyclomethycaine, dibucaine (chinchocaine), dimethocaine (larocaine), ethidocaine, hexylcaine, levobupivacaine, lidocaine, mepivacaine, meprylcaine (orocaine), metautoxycaine, piperocaine, pricocaine, procaine (novocaine), proparacaine, propoxycaine, risocaine, popicovaine, tetracaine (amethocatine), trimencaine, fatty acid amides, 12-lipoxygenase-derivated eicosanoids, black pepper compounds, other pepper components, vannilloids (n-vallinyl-alkanedienamides, N-vanillyl-alkanedienyls, N-vanillyl-cis-monosaturated alkenamides, capsiate, dihydrocapsiate, nordihydrocapsisates, capsinoids, capsiconiate, dihydrocapsiconiates, coniferyl esters, a capsiconinoid, a ricinoleic acid derivative, tinyatoxin, civamide, N-[4-(2-aminoethoxy)-3-methoxyphenyl)-methyl]-9Z-octa-decanamide, triprenyl phenols, piperines, zingerone, NE-21610, and NE-28345.

11. The method according to claim 9, wherein the TRPV1 receptor agonist is administered concurrently with the vaccine.

12. The method according to claim 9, wherein the TRPV1 receptor agonist is administered topically in the same general body area of the subject as the vaccine.

13. The method according to claim 9, wherein the TRPV1 receptor agonist is administered systemically.

14. The method according to claim 9, wherein the vaccine immunizes against measles, hepatitis B, meningitis, mumps, pertussis, poliomyelitis, rubella, tetanus, tuberculosis, yellow fever, influenza, or COVID-19.

15. The method according to claim 9, wherein the subject is a human.

16. The method according to claims 9, wherein the subject is a non-human animal.

17. The method according to claim 1, wherein the TRPV1 receptor agonist is administered after administration of the vaccine.

18. The method according to claim 3, wherein the TRPV1 receptor agonist is administered after administration of the vaccine.

19. The method according to claim 9, wherein the TRPV1 receptor agonist is administered after administration of the vaccine.

20. The method according to claim 11, wherein the TRPV1 receptor agonist is administered after administration of the vaccine.

21. A method of treating an allergic or autoimmune disorder in a subject in need thereof, which comprises administering to said subject a therapeutically effective amount if a TRPV1 receptor antagonist.

22. The method according to claim 21, wherein the TRPV1 receptor antagonist is selected from the group consisting of capsazepine (N-[2-(4-chlorophenyl)ethyl]-7,8-dihydroxy-1,3,4,5-tetrahydro-2H-benzazepine-2-carbothioamide), azo-capsazepines (AC4), thio-BCTC, BCTC, and ruthenium red.
